(19) United States
(12) Patent Application Publication
(10) Pub. No.: US 2026/0315494 A1
(43) Pub. Date: Oct. 15, 2026

(54) TREATMENT OF BREAST TISSUE

(71) Applicant: Havah Therapeutics Pty Ltd, North Adelaide (AU)

(72) Inventor: Stephen Nigel BIRRELL, Piccadilly (AU)

(21) Appl. No.: 19/484,737

(22) PCT Filed: May 17, 2024
(86) PCT No.: PCT/AU2024/050498

§ 371 Date: Nov. 14, 2025

(30) Foreign Application Priority Data
May 17, 2023 (AU) 2023901529
May 26, 2023 (AU) 2023901658

(51) Int. Cl. A61K 31/568; A61K 31/4196; A61K 31/437; A61K 31/5685; A61P 5/26; A61P 35/00

(52) U.S. Cl. CPC A61K 31/568; A61K 31/4196; A61K 31/437; A61K 31/5685; A61P 5/26; A61P 35/00

(57) ABSTRACT

There is described a method for prophylaxis or treatment of an aberrant breast cell pathology characterized by cell proliferation in a subject. The method comprises administering an effective amount of an androgenic agent in combination with an effective amount of an aromatase inhibitor to the subject providing an increase in concentration of the androgenic agent in blood serum to a level for increasing intramammary concentration of an androgen in the breast arising from action of 5α-reductase enzyme on the androgenic agent, the increased intramammary concentration of the androgen inducing suppression of c-Myc and/or mTOR expression or activity in the breast associated with a lower intramammary concentration of the androgen. The increase in the serum concentration of the androgenic agent and increase in intramammary concentration of the androgen is maintained for a predetermined period substantially without increasing concentration of the androgen in the serum of the subject. There is also described the use of the androgenic agent and aromatase inhibitor as above for reducing risk of breast cancer progressing to hormone resistant breast cancer, reducing risk of HER2-positive breast cancer becoming insensitive to targeted HER-2 anti-cancer drug therapy, and for inducing suppression of c-Myc and/or mTOR expression and/or activity. In embodiments, the androgenic agent can be testosterone and the androgen 5α-dihydrotestosterone (DHT).

### FIELD OF THE INVENTION

The present disclosure relates to a method for prophylaxis or treatment of an aberrant breast cell pathology in a subject involving treatment of the subject with an androgenic agent in combination with an aromatase inhibitor.

### BACKGROUND OF THE INVENTION

Globally, breast cancer is the most common malignancy in women. The phases of breast cancer development are initiation of the primary malignant cellular mechanism to a poorly controlled state of proliferation and undifferentiation, promotion of established malignant cells, and advancement to resistance to hormonal regulation. Attempts at prevention of breast cancer and the most frequently used therapy in breast cancer is manipulation of the inherent hormonal sensitivity of most breast cancers. Unfortunately, the most common cause of death from breast cancer is tumor growth despite anti-estrogen therapy and development of resistance to hormonal therapy.

HER2-positive breast cancers occur in nearly 20% of breast cancer cases and is associated with increased aggressiveness, poor prognosis and shortened overall survival. Drug resistance in these cancers remains a continuing challenge and molecular mechanisms that are involved in the development of resistance to Her2-targeted therapies include the PI3K/Akt/mTOR pathway (Elshazy A M and Gewitz D A, 2022).

Hormonal resistance in breast cancer refers to the phenomenon whereby breast cancer cells become resistant to hormonal therapy, which is a common treatment for hormone receptor-positive breast cancers. Hormonal therapy targets the estrogen receptor (ER) or progesterone receptor (PR) pathways to slow or stop the growth of cancer cells that rely on these hormones. There are several reasons why hormonal resistance may occur:
Genetic mutations: Alterations in genes responsible for hormone production or signaling can result in resistance to hormonal therapy. For example, mutations in the ESRI gene, which encodes the estrogen receptor, may cause resistance to anti-estrogen drugs.
Changes in receptor expression: Some breast cancer cells may stop expressing hormone receptors or express them at lower levels, making them less responsive to hormonal therapy.
Activation of alternative signaling pathways: Cancer cells may activate alternative pathways that promote cell growth and survival, bypassing the need for hormone signaling. For example, the overexpression or activation of growth factor receptors such as the HER2/neu receptor can contribute to hormonal resistance.
Crosstalk between signaling pathways: The complex interaction between different cellular pathways can also contribute to resistance. For instance, signaling molecules from other pathways can enhance the function of hormone receptors, making them less dependent on hormones.
Changes in the tumor microenvironment: The microenvironment, including factors such as surrounding cells, blood vessels, and immune cells, can influence the response of cancer cells to hormonal therapy. Some factors secreted by the tumor microenvironment may contribute to resistance.
Epigenetic changes: Epigenetic modifications, such as DNA methylation and histone modification, can regulate gene expression and alter the responsiveness of cancer cells to hormonal therapy.

As these hormonal systems are intrinsic to normal female physiological functioning there is an inevitable global hormonal side-effect associated with this therapy, which needs to be administered over a long-time frame. There has, therefore, been on ongoing need for a therapeutic or treatment that can inhibit initiation of carcinogenesis, promotion of the malignant phenotype, and inhibit hormonal resistance to therapy whilst not inducing adverse hormonal physiological events that limit the application of the therapeutic or treatment to long-term therapy.

Since the late 1800s it has been known that removal of the ovaries of a woman with advanced breast cancer could result in a good therapeutic response. In the subsequent 130 years the focus has been on establishing therapeutics which reduce the impact of the known stimulation of hormonally sensitive breast cancers by estrogen. During this time there has been interspersed activities investigating whether agonism rather than antagonism as an alternate hormonal therapy could be utilized. High dose androgens and high dose estrogens have both been demonstrated to be efficacious in the treatment of advanced metastatic hormonally sensitive breast cancer, but the side-effects have been so severe as to limit their usage. Thus, more recently and particularly in the last 20 years, there has been a proliferation of therapeutics which seek to block the effect of estrogen and overcome the development of hormonal resistance which inevitably occurs and leads to advanced breast cancer.

The mechanism behind how high dose androgens worked in the treatment of breast cancer was thought to be secondary to hypothalamic pituitary ovarian axis blockade. However, this therapy worked as efficaciously in women who had had their pituitary removed as a surgical therapeutic option and then received administered androgens upon reoccurrence of the disease as efficaciously as those patients with an intact pituitary.

Indeed, there has been significant ambiguity as to whether the major physiological androgen testosterone (T) was beneficial in breast cancer or if it was an inducer of malignancy. This has predominantly been since testosterone can be metabolized to either 5α-dihydrotestosterone (DHT) or estradiol (E), dependent on the presence and level of the enzymes 5α-reductase and aromatase. This has led to the remarkable situation where both antagonists and agonists of androgen receptor (AR) function are being trialed in the management of advanced breast cancer.

The AR is almost universally present in hormonally sensitive invasive breast carcinoma and its precursors ductal carcinoma in situ, lobular carcinoma in situ and atypical ductal hyperplasia, as well as in normal breast tissue.

Much of the bias against androgen treatment in breast cancer has arisen from the known effect of testosterone in prostate cancer (PCa) promotion. However, it has also been reported that when stimulated with high levels of androgens, the AR can function as a tumour suppressor in PCa cells, and high-dose testosterone is undergoing clinical trials for treatment of castration-resistant prostate cancer (CRPC) (Han W. et al., 2022). Those authors demonstrated that retinoblastoma (Rb) family proteins play a central role in maintaining the repressive effect of AR and that Rb inactivation desensitizes CRPC to the high-dose testosterone treatment. Those authors further showed that the efficacy of high-testosterone treatment is enhanced by a CDK4/6 inhibitor and proposed that their study demonstrated strong mechanistic and preclinical evidence for combining high testosterone dosing with CDK4/K inhibitors for the treatment of CRPC. A study of the molecular effects of high dose androgen (testosterone) on the breast tissue of transgender men (women transitioning to a male phenotype) showed breast ductal structures shrunk, testosterone induced gender-biased transcriptional changes and reshaping of the stromal compartment of the breast. The observed changes were reported to be induced directly through the AR or indirectly through paracrine signaling to cells lacking hormone receptors (Raths F. et al., 2023).

Recently, it has been reported that the androgen receptor forms an AR dimer on PCa cells in the presence of elevated DHT levels and that the dimer is associated with a reduction in c-Myc expression whereas AR exists as a monomer on PCa cells in the presence of low level DHT, and that the expression of the monomeric form of AR is associated with elevated mTOR activity in the cells (Androgen Receptor in Breast Cancer Prostate Cancer Symposium, Dec. 5, 2022 UCSF; Qiu X. et al., 2022). Another study utilising human explant tissue from breast cancers and breast cancer cell lines has demonstrated that DHT can activate endogenous AR pathways to inhibit ER-mediated transcriptional activity to those tumors that are resistant to current forms of endocrine therapy and those with genomic aberrations of ESRI or CCND1. It has also been demonstrated that when radioactive DHT was injected into women prior to undergoing breast cancer surgery that the radioactive DHT initially accumulated in the fat of the breast then strongly accumulated in the malignancy.

Doses of 500-1800 mg of testosterone have conventionally been administered in the treatment of breast cancer. High dose testosterone and aromatase inhibitor combination therapy administered by subcutaneous implant to the breast has also previously been recommended for the treatment of breast cancer with the implant dosage of testosterone typically ranging from approx. 130 mg to 200 mg or more with examples including testosterone and letrozole subcutaneous pellet implants at a dosage of 320 mg testosterone and 24 mg letrozole being administered every 9 weeks for the treatment of metastatic breast disease, with resultant serum levels of testosterone in the male range of over 20 nmol/l (i.e., 20 nM) being reported (Glaser R and Dimitrakakis C, 2021).

However, significant increases in testosterone serum levels are associated with the risk of undesirable virilization in women when administered over prolonged periods as is necessary in the treatment of cancer. Likewise, the administration of high dose aromatase inhibitor over prolonged periods can likewise have undesirable physiological effects.

International patent application WO 2016/061615 describes the administration of testosterone in combination with an aromatase inhibitor (Ai) such as anastrozole to breast tissue by way of a subcutaneous implant to provide a unique pharmacokinetic response in normal, non-malignant breast tissue in the presence of high mammographic breast density (MBD) and high magnetic resonance imaging (MRI) background parenchymal enhancement (BPE). In particular, the testosterone and anastrozole treatment was found to result in stabilisation of AR expression, elevation of serum gross cystic disease fluid protein 15 (GDFP-15) an androgen induced protein associated with a favourable prognostic outcome, and to obtain a reduction in MBD and BPE.

There remains an ongoing need for a therapeutic and treatment for breast tissue hyperplasia and cancer while reducing or avoiding undesirable effects associated with the administration of high dose testosterone and aromatase inhibitor.

### SUMMARY OF THE INVENTION

The present disclosure stems firstly from the finding that the administration of an androgenic agent in combination with an aromatase inhibitor (Ai) for the prophylaxis or treatment of breast cancer can result in a well-tolerated elevation of serum testosterone with substantially no physiologically relevant change in serum DHT level and secondly, the showing that low levels of DHT in breast tissue is associated with elevated mTOR activity whilst exposure to higher levels of DHT can result in suppression of c-Myc and/or mTOR expression/and or activity in breast tissue. The suppression of c-Myc in breast tissue is not observed with exposure to low level DHT concentration. Thus, by employing dose specific combination therapy as described herein, the risk of developing invasive breast cancer or pre-invasive malignancy of the breast may be reduced as may the risk of progression to, or genesis of, hormonally insensitive breast cancer, either by the combination treatment alone or by the treatment in conjunction with other therapies targeting the hormonal sensitivity of breast cancers. Similarly, the risk of progression to breast cancer that is insensitive to targeted HER2 therapies resulting from resistance that may occur as a result of the sustained activation of signaling pathways despite HER2 blockade, may also be reduced.

In arriving at the present disclosure, it is believed by the present inventor that the recent showing of AR dimer formation in PCa cells exposed to elevated DHT levels is responsible for the tumor suppressor activity reported in the studies of Han W. et al., 2022 and Hickey T E. et al., 2021 and that this suppression is lost with AR monomer expression at lower levels of DHT concentration. The combination treatment of an androgen and an aromatase inhibitor as described herein allows for the provision of an environment in breast tissue in which DHT or other androgen arising from 5α-reductase activity in the breast is present at a level for promoting the tumour suppressor activity of AR associated with AR dimer formation substantially without increasing systemic serum concentration of the androgen and the risk of virilization of the subject.

More particularly, in an aspect of the present disclosure there is provided a method for prophylaxis or treatment of an aberrant breast cell pathology characterized by cell proliferation in a subject, the method comprising administering an effective amount of an androgenic agent in combination with an effective amount of an aromatase inhibitor to the subject providing an increase in concentration of the androgenic agent in blood serum to a level for increasing intramammary concentration of an androgen in the breast arising from action of 5α-reductase enzyme on the androgenic agent, the increased intramammary concentration inducing suppression of c-Myc and/or mTOR expression and/or activity associated with a lower intramammary concentration of the androgen, and wherein the increase in the serum concentration of the androgenic agent and increase in intramammary concentration of the androgen is maintained for a predetermined period substantially without increasing concentration of the androgen in the serum of the subject.

Typically, the aromatase inhibitor blocks aromatization of the androgenic agent to an oestrogen. In preferred embodiments, there is substantially no increase in oestrogen concentration in the breast during the prolonged period and desirably, substantially no increase in oestrodiol concentration in the breast during the prolonged period.

Typically, the androgenic agent is selected from testosterone and physiologically active forms of testosterone.

In preferred embodiments in accordance with the present disclosure, the androgen is 5α-dihydrotestosterone (DHT) or a physiologically active form of DHT.

Typically, the intramammary concentration of the androgen is raised to above 1.0 nM.

Typically, the concentration of the androgen in the serum of the subject remains below 0.3 ng/ml (0.09 nM) over the predetermined period and most usually, is zero or negligible.

Similarly to the finding in prostate cancer (PCa) that androgen receptor (AR) can be expressed in monomer and dimer forms, it is believed by the present inventor that in methods as described herein the androgen receptor (AR) is primarily expressed as an AR monomer in the breast below the lower intramammary concentration of the androgen and primarily in AR dimer form above the lower intramammary concentration of the androgen.

In a further aspect of the present disclosure there is provided a method for method for prophylaxis or treatment of an aberrant breast cell pathology characterized by cell proliferation in a subject, the method comprising administering an effective amount of an androgenic agent in combination with an effective amount of an aromatase inhibitor to the subject providing an increase in concentration of the androgenic agent in blood serum to a level for raising intramammary concentration of 5α-dihydrotestosterone (DHT) or a physiologically active form of DHT to above 1.0 nM for a predetermined period, the increase in the concentration of the DHT or physiologically active form thereof arising from the action of 5α-reductase activity on the androgenic agent, and wherein the increase in serum concentration of the androgenic agent and increase in concentration of intramammary DHT or physiologically active form thereof is maintained for the predetermined period substantially without increasing concentration of DHT or the physiologically active form thereof in the serum of the subject over the predetermined period.

Typically, the method in accordance with the present disclosure is a method for prophylaxis or treatment of breast cancer.

Typically, the breast cancer is pre-invasive cancer or malignant breast cancer. The pre-invasive breast cancer in at least some forms of the present disclosure can be ductal or lobular cell carcinoma in-situ (DCIS or LCIS).

In other embodiments, the method is for prophylaxis or treatment of breast cell hyperplasia and typically, ductal or lobular cell hyperplasia.

Particularly desirably, the risk of the breast cancer or breast hyperplasia progressing to hormonal resistant breast cancer or breast cancer insensitive to targeted HER2 anti-cancer drug therapy may be reduced by the administration of the androgenic agent and aromatase inhibitor combination therapy as described herein.

In particular, in accordance with still another aspect of the present disclosure there is provided a method for reducing risk of breast cancer in a subject progressing to hormone resistant breast cancer, the method comprising administering an effective amount of an androgenic agent in combination with an effective amount of an aromatase inhibitor to the subject providing an increase in concentration of the androgenic agent in blood serum to a level for increasing intramammary concentration of the androgen arising from action of 5α-reductase enzyme on the androgenic agent, the increased intramammary concentration inducing suppression of c-Myc and/or mTOR activity in the breast associated with a lower intramammary concentration of the androgen, and wherein the increase in serum concentration of the androgenic agent and increase in intramammary concentration of the androgen in the breast is maintained for a predetermined period substantially without increasing of concentration of the androgen in the serum of the subject.

In another aspect of the present disclosure there is provided a method for reducing risk of HER2-positive breast cancer in a subject becoming insensitive to targeted HER2 anti-cancer drug therapy, the method comprising administering an effective amount of an androgenic agent in combination with an effective amount of an aromatase inhibitor to the subject providing an increase in concentration of the androgenic agent in blood serum to a level for increasing intramammary concentration of the androgen arising from action of 5α-reductase enzyme on the androgenic agent, the increased intramammary concentration inducing suppression of c-Myc and/or mTOR activity in the breast associated with a lower intramammary concentration of the androgen, and wherein the increase in serum concentration of the androgenic agent and increase in intramammary concentration of the androgen in the breast is maintained for a predetermined period substantially without increasing concentration of the androgen in the serum of the subject.

In another aspect of the present disclosure there is provided a method for inducing suppression of c-Myc and/or mTOR activity in the breast of a subject, the method comprising administering an effective amount of an androgenic agent in combination with an effective amount of an aromatase inhibitor to the subject providing an increase in concentration of the androgenic agent in blood serum and an increase in intramammary concentration of an androgen arising from action of 5α-reductase enzyme on the androgenic agent, the increased intramammary concentration inducing the suppression of c-Myc and/or mTOR activity in the breast associated with a lower intramammary concentration of the androgen.

In another aspect of the present invention there is provided an androgenic agent for use in a method for the prophylaxis or treatment of breast cancer or breast cell hyperplasia in a subject, the method comprising administering an effective amount of an androgenic agent in combination with an effective amount of an aromatase inhibitor to the subject providing an increase in concentration of the androgenic agent in blood serum to a level for increasing intramammary concentration of an androgen in the breast arising from action of 5α-reductase enzyme on the androgenic agent, the increased intramammary concentration inducing suppression of c-Myc and/or mTOR activity in the breast associated with a lower intramammary concentration of the androgen, and wherein the increase in the serum concentration of the androgenic agent and increase in intramammary concentration of the androgen is maintained for a predetermined period substantially without increasing concentration of the androgen in the serum of the subject.

In another aspect of the present invention there is provided the use of an androgenic agent in the manufacture of a medicament for the prophylaxis or treatment of breast cancer or breast cell hyperplasia in a subject, the prophylaxis or treatment comprising administering an effective amount of an androgenic agent in combination with an effective amount of an aromatase inhibitor to the subject providing an increase in concentration of the androgenic agent in blood serum to a level for increasing intramammary concentration of an androgen in the breast arising from action of 5α-reductase enzyme on the androgenic agent, the increased intramammary concentration inducing suppression of c-Myc and/or mTOR activity in the breast associated with a lower intramammary concentration of the androgen, and wherein the increase in the serum concentration of the androgenic agent and increase in intramammary concentration of the androgen is maintained for a predetermined period substantially without increasing concentration of the androgen in the serum of the subject.

In a further aspect of the present disclosure there is provided an androgenic agent for use in a method for prophylaxis or treatment of breast cancer or breast cell hyperplasia in a subject, the method comprising administering an effective amount of an androgenic agent in combination with an effective amount of an aromatase inhibitor to the subject providing an increase in concentration of the androgenic agent in blood serum to a level to raise intramammary concentration of dihydrotestosterone (DHT) or a physiologically active form of DHT to above 1.0 nM for a predetermined period, the increase in the concentration of the DHT or physiologically active form thereof arising from the action of 5α-reductase activity on the androgenic agent, and wherein the increase in serum concentration of the androgenic agent and the increase in intramammary concentration of DHT or physiologically acceptable form thereof is maintained for the predeterminer period substantially without increasing concentration of DHT or the physiologically active form thereof in serum of the subject over the predetermined period.

In a yet further aspect of the present disclosure there is provided the use of an androgenic agent in the manufacture of a medicament for prophylaxis or treatment of breast cancer or breast cell hyperplasia in a subject, the prophylaxis or treatment comprising administering an effective amount of an androgenic agent in combination with an effective amount of an aromatase inhibitor to the subject providing an increase in concentration of the androgenic agent in blood serum to a level to raise intramammary concentration of dihydrotestosterone (DHT) or a physiologically active form of DHT to above 1.0 nM for a predetermined period, the increase in the concentration of the DHT or physiologically active form thereof arising from the action of 5α-reductase activity on the androgenic agent, and wherein the increase in serum concentration of the androgenic agent and the increase in intramammary concentration of DHT or physiologically acceptable form thereof is maintained for the predeterminer period substantially without increasing concentration of DHT or the physiologically active form thereof in the serum of the subject over the predetermined period.

Thus, methods for the treatment of breast cell hyperplasia, pre-invasive and malignant breast cancers by agonist activation of the androgen receptor (AR) are described. In embodiments, the intramammary concentration of the androgen is increased above a lower limit to induce or promote tumour suppressor activity of AR resulting in suppression of the expression and/or activity of the oncogene c-Myc, and suppression, avoidance or a reduction in mTOR expression and/or activity, associated with an intramammary concentration of the androgen below a lower limit. Embodiments as described herein therefore provide for prophylaxis or treatment in a dose specific fashion. The effect of the increased intramammary concentration of androgen is believed to arise from the formation of AR dimer at the elevated concentration of the androgen compared to expression of AR monomer at lower concentration of the androgen in the breast.

Accordingly, the present disclosure allows for the induction of suppression of c-Myc and/or mTOR expression or activity, inhibiting cellular proliferation and providing for induction of a differentiated phenotype. Further, in one or more forms, the present disclosure may allow for inhibiting, blocking or reducing the risk of progression to drug resistant breast cancer e.g., hormonal resistance breast cancer or HER2-positive breast cancer insensitive to targeted HER2 anti-cancer therapies, and all such methods are expressly contemplated.

Advantageously also, in preferred embodiments as described herein, the concentration of the administered androgenic agent in the blood serum of the patient remains at level that substantially does not result in virilization or visual change in physical characteristics of the subject typically associated with the androgenic agent during the predetermined period, or wherein any such virilization or change is essentially negligible. Likewise, as the level of the androgen in the breast by the action of 5α-reductase may be obtained substantially without any increase in serum concentration of the androgen, in embodiments described herein there is also essentially no virilization of the subject arising from the androgen other than the effects exerted by the androgen in the breast.

Moreover, methods of the present disclosure in one or more forms provide for treatment of breast cancer as described herein with reduced dosages of testosterone and aromatase inhibitor than have conventionally been used. By allowing for the use of lower doses of both testosterone and aromatase inhibitor to be utilized, the risk of unwanted or undesirable systemic side effects from the aromatase inhibitor may also be reduced or avoided. Further, given testosterone is a precursor to oestrogen which promotes breast cell proliferation, by providing for administration of a reduced dosage of testosterone and for relatively low increases in serum levels of testosterone as described herein, the risk of unwanted and undesirable oestrogen synthesis is also reduced. Additionally, a method in accordance with one or more forms of the present disclosure may allow for enhanced cancer therapy compared to existing conventional therapies.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or elsewhere before the priority date of this application.

The features and advantages of the present invention will become further apparent from the following detailed description of exemplary embodiments of the invention together with the accompanying drawings.

### DETAILED DESCRIPTION OF EXEMPLARY EMBODIMENTS

Deregulated expression of c-Myc and over activation of mTOR signalling is associated with initiation and progression of cancers including breast cancer.

c-Myc is a proto-oncogene that encodes the transcription factor protein MYC. MYC plays a crucial role in regulating cell growth, division, and metabolism. It can also influence cell differentiation, apoptosis (cell death), and the maintenance of stem cell-like properties in cancer cells. In breast cancer, c-Myc is often found to be overexpressed or amplified, meaning there is an increased production of the MYC protein, which can contribute to the development and progression of cancer. In breast cancer, c-Myc has several roles:
Promotes cell proliferation: Overexpression of c-Myc can enhance cell growth and division by regulating the expression of genes involved in the cell cycle, thereby accelerating the cell cycle and increasing the rate of cell proliferation.
Inhibits apoptosis: c-Myc can inhibit the process of programmed cell death (apoptosis) by downregulating the expression of pro-apoptotic genes and upregulating anti-apoptotic genes. This allows cancer cells to survive and continue to grow despite unfavorable conditions.
Increases metabolic activity: c-Myc can upregulate the expression of genes involved in metabolism, particularly those involved in glucose and glutamine metabolism. This increased metabolic activity provides the necessary energy and building blocks for the rapid growth and proliferation of cancer cells.
Contributes to tumor heterogeneity: c-Myc can influence the differentiation and maintenance of cancer stem cells, which are a subpopulation of tumor cells with the ability to self-renew and generate diverse cell types within the tumor. This contributes to tumor heterogeneity, making the cancer more aggressive and harder to treat.
Facilitates angiogenesis: c-Myc may also contribute to the formation of new blood vessels (angiogenesis) in the tumor, which is essential for providing nutrients and oxygen to the growing cancer cells.
Promotes metastasis: c-Myc can also promote the invasion and spread of cancer cells to other parts of the body by regulating genes involved in cell adhesion, migration, and extracellular matrix remodeling.

mTOR is a protein kinase that plays a central role in regulating cell growth, proliferation, survival, metabolism, and angiogenesis, and is a key component of the PI3K/AKT/mTOR signaling pathway which is frequently dysregulated in various cancers, including breast cancer. In breast cancer, the activation or overactivation of the mTOR pathway contributes to cancer development and progression in several ways:
By enhancing cell proliferation and survival: Activation of the mTOR pathway promotes cell growth and division by regulating protein synthesis, ribosome biogenesis, and cell cycle progression. Additionally, mTOR signaling can inhibit apoptosis, allowing cancer cells to survive and continue to grow.
By increasing metabolism and nutrient uptake: mTOR plays a role in regulating cellular metabolism, including glucose and lipid metabolism, to meet the energy and biosynthetic demands of rapidly dividing cancer cells. The activation of the mTOR pathway can also increase nutrient uptake, supporting cancer cell growth and survival.
By contributing to tumor angiogenesis: mTOR signaling can promote the formation of new blood vessels (angiogenesis) within tumors by upregulating the expression of pro-angiogenic factors such as vascular endothelial growth factor (VEGF). This ensures that the growing tumor receives adequate nutrients and oxygen.
By affecting cancer cell migration and invasion: Activation of the mTOR pathway can influence the expression of proteins involved in cell adhesion, migration, and extracellular matrix remodeling, which can facilitate cancer cell invasion and metastasis.
By supporting cancer stem cells: mTOR signaling is implicated in the maintenance and self-renewal of cancer stem cells, a subpopulation of tumor cells with the ability to generate diverse cell types within the tumor. This contributes to tumor heterogeneity and can make cancer more aggressive and harder to treat.

By suppressing c-Myc and sustained activation of signaling pathways such as the PI3K/Akt/mTOR pathway as described herein, initiation and progression to hormone resistant breast cancer or drug resistance cancer such as HER2-positive cancer insensitive to targeted HER2 anti-cancer drug therapy, despite HER2 blockade, may be slowed and/or the risk of initiation or progression of breast cancer reduced. c-Myc for instance has been reported to regulate the sensitivity of breast cancer cells to the selective CDK4 and CDK6 inhibitor palbociclib via the c-Myc/miR-29b-3p/CDK axis and inactivation of c-Myc can increase palbciclib efficacy (Ji W. et al, 2020).

Methods of the present disclosure stem from the observation that c-Myc and mTOR signaling and/or activity can be suppressed or blocked in the breast when the intramammary concentration of 5α-dihydrotestosterone (DHT) is increased above a lower limit of 1 nM. Without being limited by theory, it is believed by the inventor that the increase in intramammary DHT level induces AR dimer formation and transcriptional activation of androgen receptor (AR) dimer resulting in suppression of c-Myc and/or mTOR activity found with an intramammary DHT concentration below 1 nM, and wherein this c-Myc and mTOR activity is associated with expression of AR in monomer form below a DHT intramammary concentration of 1 nM.

Breast cancer in men is rare and when it does occur the tumour is generally positive for androgen receptor (AR) expression and hormone receptor positive for oestrogen and progesterone. Male breast malignancy likely occurs secondarily to a genetic predisposition but despite this the malignancy typically maintains hormonal sensitivity which would suggest a better response to therapy. Men are more likely to have elevated DHT in their breast tissue and this is the most likely reason why men do not normally develop breast cancer. Under normal circumstances a woman does not have elevated DHT level in her breast tissue and as a result, AR receptor-induced downstream genetic alterations in breast tissue associated with elevated DHT will generally not occur.

Recently, it has been reported that the androgen receptor (AR) can interact with the DNA binding factor GATA3 (GATA-binding protein 3) and that DHT induces AR/GATA3 binding facilitating the tumour suppressor function of AR, wherein GATA3 binding to the AR is associated with AR-mediated growth inhibition in breast cancer cells and better overall breast cancer survival prognosis, regardless of oestrogen receptor (ER) expression status of the cells (Hosseinzadeh, L. et al., 2024).

In embodiments, an increase in DHT (or other androgen) level in the breast arising from 5α-reductase activity as described herein may promote the tumour suppressor activity of AR associated with AR dimer formation, and so GATA3 binding to the AR. As GATA3 binding to the AR receptor has recently been demonstrated to facilitate AR tumour suppressor activity (Hosseinzadeh, L. et al., 2024) and thereby be a critical element associated with breast cells which are least likely to have malignant progression or become hormonally insensitive, but which are able to be treated with a hormonal therapy, the induction of the tumour suppressor activity of the AR provides for inhibition or prevention of the formation or progression to ER negative breast cancer, and treatment with an androgenic agent and aromatase inhibitor as described herein may provide a therapeutic treatment for reprogramming of ER negative breast cancer cells or tumors to hormonally sensitive breast cancer cells or tumors (e.g., cancers which express ER and/or progesterone receptor (PR), and/or which are treatable with hormone therapies as for instance described herein), and all such embodiments are expressly contemplated.

In particular, in at least some forms the present disclosure provides for the inhibition, prevention or reduced risk of progression to hormonally sensitive or hormonally insensitive breast cancer, or if malignant breast cancer arises, that the cancer is hormonally sensitive with an improved prognosis and/or therapeutic response to hormonal treatment.

Testosterone is converted to DHT by the action of 5α-reductase, an enzyme that is present in the breast and the expression of which has been reported to be upregulated by DHT. While testosterone is typically administered in accordance with a method of the present disclosure, other androgenic agents that can be reduced by 5α-reductase in the breast can also be utilized. DHT itself is an agonist of the androgen receptor (AR) for which it has 2 to 3 times higher affinity than testosterone, and has been reported to be 2.5 to 10 times more potent than testosterone. Testosterone is also converted to oestradiol in the breast by aromatase enzyme. By administering testosterone or other androgenic agent as described herein to a subject in preferred embodiments of the present disclosure in combination with an aromatase inhibitor, the concentration of DHT can be increased in the breast without deleteriously increasing breast oestradiol, to thereby obtain an antiestrogenic effect in breast tissue.

By “androgenic agent” as used herein is meant testosterone and other forms of steroid and non-steroid hormones that bind to the androgen receptor (e.g., in at least the nM range) to stimulate transactivation activity and thus regulate the expression of target genes, and which can be 5α-reduced by 5α-reductase enzyme in the breast of the subject, and includes synthetic and non-synthetic hormones, and physiologically active forms of testosterone, non-limiting forms of which include precursors of testosterone, and derivatives, isomers and esters of testosterone.

Examples of androgenic agents include methyltestosterone which is a synthetic form of testosterone with a methyl group at the 17th carbon position and can be reduced by 5α-reductase to form 17α-methyldihydrotestosterone (17α-MDHT); fluoxymesterone, another synthetic androgen that can be converted to dihydrofluoxymestrone (DHF); metandienone (also known as methandrostenolone) a steroid derived from testosterone which can be reduced to dihydromethanrostenolone (also known as 17α-methyl-5α-dihydrotestosterone or 17α-methyl-DHT); androstenedione, a precursor of testosterone which can be converted to 5α-androstanedione by 5α-alpha reductase; and dehydroepiandrostone (DHEA) which can be converted by 5α-alpha reductase to 5α-androstanediol. Further physiologically acceptable forms of testosterone that may be utilized in a method herein include testosterone enanthate, testosterone propionate, testosterone cypionate and testosterone undecanoate.

By “androgen” as used herein in the context of effecting prophylaxis or treatment of the present disclosure is meant the androgen arising from the action of 5α-reductase on the androgenic agent in the breast of the subject and which binds to the androgen receptor, and includes DHT and physiologically active forms of DHT.

By “aromatase inhibitor” as used herein is meant an inhibitor of aromatase enzyme for blocking aromatization of the androgenic agent into an oestrogen, such as oestradiol.

An aromatase inhibitor for use in a method of the present disclosure may be selected from non-steroidal aromatase inhibitors, steroidal aromatase inhibitors and mechanism-based steroidal inhibitors. Non-steroidal aromatase inhibitors can be divided into three classes: aminoglutethimide-like molecules, imidazole/triazole derivatives, and flavonoid analogs. Examples of non-steroidal aromatase inhibitors include, but are not limited to, anastrozole (Arimidex®; 2,2′-[5-(1H-1,2,4-triazol-1-ylmethyl)-1,3-phenylene]bis(2-methylpropanenitrile)); letrozole (Femara®; (4,4′-((1H-1,2,4-triazol-1 yl)methylene)dibenzonitrile); vorozole (Rivizor®) and fadrozole (Afema®).

Exemestane (Aromasin®; (6-methylenandrosta-1,4-diene-3,17-dione)) and formestane (Lentaron®; 4-hydroxyandrost-4-ene-3,17-dione)) are examples of steroidal aromatase inhibitors that may be employed. Typically, the aromatase inhibitor used in a method of the present disclosure is selected from the group consisting of anastrozole and letrozole and most usually, is anastrozole.

By “effective amount” or “pharmaceutically effective amount” as used herein is meant a sufficient amount of the active compound or drug to provide the desired therapeutic effect and is non-toxic, an acceptable toxicity profile and/or an acceptable side effects profile. The amount may vary from subject to subject depending on, for example, age, general physiological condition of the subject, the severity of the condition being treated, the particular therapeutic agent being to be administered, and combinations of these factors. An appropriate “effective amount”, typically in an individual case, may be determined by the attending physician or medical professional by reference to pertinent texts and literature and/or using routine experimentation and evaluation according to well established principles.

The dosage of the selected aromatase inhibitor will generally be at a level determined to avoid intramammary increase in concentration of naturally occurring or synthetic oestrogens arising from the administered androgenic agent, and particularly oestradiol which is well known to promote breast epithelial and stroma cell proliferation and differentiation.

In methods described herein, the androgenic agent is administered to the subject to result in an increase in blood serum concentration of the androgenic agent to a level corresponding to an increase in intramammary concentration of the androgen arising from the action of 5α-reductase on the androgenic agent in the breast that is sufficient to obtain or maintain suppression of c-Myc and/or mTOR in the breast. The suppression of c-Myc and/or mTOR activity may at least be a majority of c-Myc and/or mTOR activity (e.g., greater than 50%, 55%, 60%, 65%, 70%, 75%, 80%, 85%, 90%, 95%, 96%, 97%, 98% or 99% or more), or may be complete or substantially complete whereby any detectable c-Myc and/or mTOR activity is negligible or below a physiologically relevant level, as may be determined by a polymerase chain reaction (PCR) protocol, ELIZA, cell assay or other appropriate assay.

The suppression of c-Myc may be embodied by downregulated or blocked activation of the c-Myc oncogene and/or down-regulated expression and/or activity of the encoded MYC protein. Likewise. Likewise, the suppression of mTOR can comprise down-regulated or blocked expression and/or activity of the kinase.

Aberrant breast cell pathologies that may be treated in accordance with the present disclosure include breast cell hyperplasia such as atypical breast cell hyperplasia, ductal cell hyperplasia and lobular cell hyperplasia, ductal cell carcinoma in-situ (DCIS), lobular cell carcinoma in-situ (LCIS), carcinoma, pre-invasive cancer and malignant cancer of the breast. By pre-invasive cancer is meant a cancer or carcinoma that while involving unregulated cell proliferation, has not spread or metastasized from its site of origin to surrounding breast structures or from ducts, lobules or glands of the breast. In contrast, a malignant cancer is one that has metastasized to other area(s) of the breast or body of the subject, or has spread from a duct, lobule or gland of the breast to surrounding breast tissues.

DCIS is a non-invasive form of breast cancer where abnormal cells are found in the lining of a breast duct but have not spread beyond the duct, and is considered the earliest form of breast cancer. DCIS is highly treatable but can progress to invasive breast cancer if not managed appropriately.

GATA3 is a transcription factor that plays a crucial role in the development and differentiation of various tissues including the mammary gland. It is one of the key regulators of luminal epithelial cell differentiation in the breast and is involved in maintaining the function and integrity of these cells. GATA3 is a marker for luminal epithelial cells in the breast and its expression in DCIS indicates that the tumor cells have luminal characteristics, which is typical of many breast cancers, especially those classified as luminal A or luminal B subtypes. This marker is therefore widely used in pathology to assist differentiation of various types of breast lesions and its presence can assist in confirming a diagnosis of DCIS and distinguish it from other breast pathologies, including invasive carcinoma and benign conditions. Studies have shown that GATA3 expression is associated with favorable prognostic factors in breast cancer, and high levels of GATA3 expression in DCIS may be correlated with a lower risk of progression to invasive disease and better overall prognosis. Further, the role of GATA3 in regulating estrogen receptor (ER) signaling pathways makes it relevant for treatment of the patient with hormonal therapy. For instance, tumors expressing GATA3 are often ER-positive, which means they may respond well to anti-estrogen therapies like tamoxifen or aromatase inhibitors. Further, GATA3 influences cell proliferation, differentiation, and apoptosis which are critical processes in cancer development, progression and response to treatment. GATA3 is therefore a significant biomarker in the context of DCIS, providing diagnostic, prognostic, and therapeutic insights. Luminal breast cell differentiation with GATA3 expression is associated with a favorable prognosis and responsiveness to hormonal therapies as discussed above, and the present disclosure extends to determination of the induction of GATA3 expression and/or activity by treatment with an androgenic agent in combination with aromatase inhibitor as described herein for evaluation of prognosis and/or selection or suitability of cancer cell treatment (e.g., by hormonal therapy such as tamoxifen or other hormonal therapies described herein), which is expressly contemplated herein. For instance, elevated GATA3 expression and/or activity in a breast cell sample from a subject treated with an androgenic agent and aromatase inhibitor may be evaluated by suitable assay to determine whether or not GATA3 activity is above or below a reference level wherein expression or activity of the marker above the reference level is indicative of a more favourable prognosis and/or suitability for hormonal thereby, compared to an evaluated level of GATA3 expression and/or activity in the test sample that is below the reference level. DCIS can also be divided into two distinct sub-groups: “immune hot” and “immune cold”, the first of which is characterized by increased infiltration of lymphocytes and macrophages associated with a “hot” immune response and poorer prognosis, with elevated expression of the markers PDCD1 and CTLA4, and reduced expression of GATA3 (Hong R. et al., 2024). Mutational analysis of GATA3 in breast cancer has also shown a relatively high mutation rate in young-onset IDS in women and in male breast cancer (Hong R. et al., 2024 and Li Y. et al, 2024). Evaluation of GATA3 expression and/or activity may therefore be combined with assessment of immune response status of the cancer and/or GATA3 mutational analysis of the cell sample for evaluation of prognosis and/or selection or suitability of cancer cell treatment as described herein.

Typically, the androgenic agent when administered to the subject on accordance with the present disclosure will raise serum concentration of the androgenic agent to 0.2 ng/ml or above and usually, 0.25 ng/ml, 0.3 ng/ml, 0.35 ng/ml, 0.4 ng/ml, 0.45 ng/ml, 0.5 ng/ml, 0.55 ng/ml, 0.60 ng/ml. 0.65 ng/ml, 0.70 ng/ml, 0.75 ng/ml, 0.8 ng/ml, 0.85 ng/ml, 0.9 ng/ml. 0.95 ng/ml, 1 ng/ml, 1.25 ng/ml, 1.5 ng/ml, 1.75 ng/ml, 2 ng/ml, 2.25 ng/ml, 2.5 ng/ml, 3 ng/ml, 3.5 ng/ml, 3.75 ng/ml, 4 ng/ml, 4.25 ng/ml, 4.5 ng/ml, 5 ng/ml, 5.5 ng/ml, 6 ng/ml, 6.5 ng/ml or 7 ng/ml or higher. Most usually, the serum concentration of the androgenic agent will be increased to within a range of from about 0.2 ng/ml, 0.25 ng/ml or 0.5 ng/ml or greater to about 7 ng/ml, 6.5 ng/ml, 6 ng/ml, 5.75 ng/ml or 5.5 ng/ml or less and all serum concentrations of the androgenic agent within the above specified ranges are expressly encompassed. Typically, the serum concentration of the androgenic agent will be increased to a concentration of about 4 ng/ml to about 6 ng/ml, and typically will be about 5 ng/ml.

In embodiments, the androgenic agent will generally be administered at a dosage whereby the intramammary concentration of the androgen arising from the action of 5α-reductase in the breast is increased to a level of 1 nM or greater for obtaining the suppression of c-Myc and/or mTOR activity, as may be achieved via the induction or activation of AR mediated suppressor activity in the breast. Generally, the serum concentration of androgenic agent will be increased by the administered androgenic agent to obtain an intramammary concentration of the 5α-reduced androgen above 1 nM and most usually, to a concentration of the androgen in the breast of about 1.2 nM, 1.25 nM, 1.3 nM, 1.35 nM, 1.4 nM, 1.45 nM, 1.5 nM, 1.55 nM, 1.6 nM, 1.65 nM, 1.7 nM, 1.75 nM, 1.8 nM, 1.85 nM, 1.9 nM, 1.95 nM, 2 nM, 2.25 nM, 2.5 nM, 3 nM, 3.5 nM, 4 nM, 4.5 nM, 5 nM, 6 nM, 7 nM, 8 nM, 9 nM, 10 nM, 11 nM, 12 nM, 13 nM or 14 nM or higher, and preferably in a range from above 1 nM to about 12 nM, more usually in a range of from 1 nM to about 10 nM and most preferably, in a range of from about 1.5 nM, 1.75 nM or 2 nM or above to about 10 nM, and all intramammary concentrations of the androgen in the above specified are expressly provided for herein.

The androgenic agent is typically testosterone or a physiologically active form thereof and so the 5α-reduced androgenic agent in the breast is typically DHT or a physiologically active form of DHT. Most typically, the androgenic agent is testosterone and the androgen obtained by the action of 5α-reductase in the breast is DHT.

Where the androgenic agent is other than testosterone, the androgenic agent can be administered to the subject at a dosage to provide an increase in the intramammary concentration of the 5α-reduced form of the androgenic agent in the breast for obtaining the androgenic effect resulting in suppression of c-Myc and/or mTOR activity as described herein that is equivalent to at least the lower intramammary concentration of DHT required to obtain that effect, as may be evaluated by suitable assay(s).

The androgenic agent is typically administered to the subject at a dosage dependent on the mode of administration and which is selected to be sufficient to raise and maintain the serum concentration of the androgenic agent in a range for increasing the intramammary concentration of the androgen arising from the action of 5α-reductase on the androgenic agent in the breast above the level required to induce both the suppression of c-Myc and/or mTOR activity as described herein, for a predetermined period of at least 1 week, more usually at least 2 weeks or 3 weeks and preferably, at least 1 month, 2 months or 3 months. In particularly preferred embodiments, the increase in serum concentration of the androgenic agent is maintained by the administered dosage of the androgenic agent for a period of about 3 months. As will be understood, by maintaining the serum concentration of the administered androgenic agent elevated over the predetermined period in accordance with the present disclosure, the increased intramammary concentration of the androgen required to exert its therapeutic effects is also maintained above the level required to induce the suppression of c-Myc and/or mTOR over the predetermined period.

The dosage of the selected aromatase inhibitor administered is likewise selected to be sufficient to block aromatization of the androgenic agent in the breast and thereby oestrogen and particularly, oestradiol production in breast tissue.

Ongoing treatment of the subject can be obtained by repeat administration of the androgenic agent and aromatase inhibitor at regular intervals corresponding to the end of each predetermined period (e.g., every 3 months) as required to maintain the increased serum level of the androgenic agent and increase in intramammary concentration of the androgen resulting from 5α-reductase activity in the breast over the predetermined period or within the required range as described herein.

The androgenic agent and aromatase combination therapy of the present disclosure can be administered to the subject by any suitable method of application, such as by subcutaneous implant, transdermal delivery or by injection (e.g., subcutaneous, intramuscular and intravenous injection), typically for continuous or sustained delivery of the androgenic agent and aromatase.

Further, the androgenic agent and aromatase inhibitor can be administered simultaneously or sequentially in the same or different pharmaceutical or veterinary compositions to exert their effects in the same or overlapping therapeutic window(s). A pharmaceutical or veterinary composition useful herein can contain one or more of binders, fillers, preservatives and pharmaceutically acceptable or physiologically acceptable carriers and excipients conventionally used in pharmaceutical or veterinary formulations and preparations. The term “pharmaceutically acceptable” or “physiologically acceptable” is understood to mean those compounds, agents, materials, compositions, excipients, carriers and/or dosage forms that are, within the scope of sound medical judgment, suitable for contact with the tissues of human beings and/or animals without excessive toxicity, irritation, allergic response, or other problems or complications commensurate with a reasonable benefit and/or risk ratio.

Typically, the combination therapy is administered by subcutaneous implant (e.g., subcutaneous pellet(s)) or transdermally such as by transdermal patch, and most usually by subcutaneous pellet inserted into the lower abdomen, upper gluteal region, or other location as deemed suitable. Subcutaneous pellets useful of administration of the androgenic agent and aromatase of the present combination therapy are, for instance, described in International patent applications WO 2016/061615 and WO 2020/243777, the contents of both of which are incorporated herein in their entirety. In particular, the androgenic agent and aromatase inhibitor are preferably administered in the form of a multi-phasic composition as described in WO 2020/243777 and the use of all such compositions, dosage forms, implants and pellets as described therein are expressly encompassed.

In particularly preferred embodiments, the combination therapy is administered to the subject by subcutaneous pellet comprising testosterone in amount of from about 40 mg to about 120 mg and anastrozole in amount of from 2 mg to about 6 mg. Typically, the pellet comprises from about 60 mg to about 100 mg, and most preferably about 80 mg of testosterone or about 100 mg of testosterone, and anastrozole in an amount of from about 3 mg to about 5 mg and most preferably, about 4 mg.

When letrozole or vorozole are utilized as the aromatase inhibitor in a method as described herein, the dosage employed in the pellet is typically in the range of from 8 mg to 16 mg.

To obtain ongoing treatment of the subject, the pellet is administered to the subject every 3 months for as long as deemed appropriate.

As methods as described herein provide for maintenance of a low serum level of the administered androgenic agent substantially without an increase in serum concentration of the androgen arising from 5α-reductase activity in the breast, the subject can be treated as described herein for extended periods of months and years (e.g., 1, 2, 3 or more years), essentially without virilization of the subject and avoiding high dosages of testosterone and aromatase enzyme that have been conventionally administered in the treatment of breast cancer.

By inducing the suppression of c-Myc and/or mTOR activity as described herein over a prolonged period, the progression of the aberrant breast cell pathology to breast cancer, pre-invasive breast cancer, malignant breast cancer, hormone resistant breast cancer or e.g., HER2-positive breast cancer progressing to cancer insensitive to targeted HER2 drug therapy, despite HER2 blockade, may be slowed, blocked or the risk of progression reduced. Hormone therapy of breast cancer responsive to such therapy includes treatment with androgens, selective estrogen receptor modulators (SERMS), aromatase inhibitors, estrogen blockers, and anti-estrogen drugs that target the estrogen receptor (ER) to block or down-regulate ER signaling. SERMs are a class of drugs that interact with ER in a selective manner, examples of which include tamoxifen, raloxifene, toremifene, clomiphene, bazedoxifene and lasofoxifene. Selective estrogen receptor degraders (SERDs) are another class of drugs that target ER, and act to induce degradation of the ER, leading to a more potent and sustained inhibition of ER signaling. Examples of SERDs that may be employed include fulvestrant, GDC-0810 (Buparlisib), GDC-0927, AZD9496, elacestrant (RAD1901) and LSZ102.

Therapeutics used in targeted HER2-positive cancer therapy include monoclonal antibodies such as trastuzumab, pertuzumab, margetuximab-cmkb, antibody-drug conjugates, pan-HER inhibitors such as neratininb, tyrosine kinase inhibitors such as tucatinib, and signal transducer inhibitors such as lapatinib.

The subject can be any mammal treatable in accordance with a method of the present disclosure and may, for example, be selected from the rodent, feline, canine, equine, porcine, caprine, ovine, Leporidae, and primate animal families e.g., mice, rats, guinea pigs, hamsters, rabbits, cats, dogs, horses, domesticated animals such as goats and sheep, and monkeys, baboons and apes. Typically, the subject is female and most typically, is a human. The subject may, for example be a pre-menopausal, peri-menopausal or post-menopausal woman, though any female deemed a suitable candidate may be treated by a method as described herein. By “peri-menopausal” is meant the woman is undergoing transition toward menopause and may notice signs of this progression such as menstrual irregularity. During peri-menopause, oestrogen levels may be rise and fall unevenly, menstrual cycles may lengthen or shorten, and menstrual cycles may begin in which ovulation does no occur. Menopause-like symptoms may also be experienced, such as hot flushes, sleep problems, and mood changes and swings. A “post-menopausal” woman is one or has naturally passed through menopause, who has had their ovaries removed or whose oestrogen production has been permanently suppressed to a level so as to not have had a menstrual period for 12 months.

Methods in accordance with the present disclosure are further described below by way of non-limiting examples.

### EXAMPLES

### 1. Effect of Testosterone and Aromatase Inhibitor

### 1.2 Method

A single dose, single centre open-label and non-randomised trial of the effect of testosterone in combination with aromatase inhibitor treatment in premenopausal women was conducted at Wellend Health Clinic (Wellend Health, Nyroca House, North Adelaide, SA, Australia).

The premenopausal women were aged between 35 and 55 years of age with a BMI range of 20-30 kg/m². A single dose of a T+Ai™ pellet implant containing 80 mg of testosterone (T) and 4 mg of the aromatase inhibitor (Ai) anastrozole was inserted subcutaneously in the lower abdomen or upper gluteal region between 8 am and 10 am on the day of dosing i.e., Day 1. The implant was intended to be left in situ and was expected to be almost completely absorbed within three months following insertion.

A total of 11 subjects were dosed with the implant and included in the safety analysis set. All 11 subjects were included in the pharmacokinetics (PK) analysis set.

Serum concentrations of testosterone, dihydrotestosterone (DHT) and the plasma concentrations of anastrozole were determined at: predose, 1, 2, 4, 6, 8 and 12 hours after dosing on Day 1, and in a single sample collected on Days 2, 3, 4, 5, 8, 15, 22, 29, 43, 57, 71 and 85. (NB: the sample collection time point at 6 hours post-dose was added for the third and subsequent subject and so serum measurements were not determined for the first two subjects as noted below).

Patients seeking treatment with the T+Ai for reduction in high MBD were eligible for the study. Following successful screening, subjects attended the Wellend Health Clinic for dosing. The trial was designed to dose all subjects while in the luteal phase of their menstrual cycle to avoid the highly changing hormonal environment of the follicular phase. Following insertion of the implant, PK blood sampling was carried out for a duration of three months (see below). Briefly, samples were taken repeatedly on the day of dosing, daily during the first week, weekly for the next four weeks and every second week during the last eight weeks of a total sampling period of three months. After all assessments on Day 85 had been completed, the trial ended and subjects continued on with the testosterone and aromatase inhibitor therapy if they wished, as per the agreed clinical practice.

To determine whether the selected PK sampling schedule provided an adequate description of the serum/plasma concentration time profile of the testosterone and aromatase inhibitor therapy, after the Day 29 blood samples had been collected from the first two participants, the samples were analysed for testosterone, dihydrotestosterone and anastrozole. The sampling schedule was revised with the addition of one blood sample (at 6 hours post-dose as discussed above), and no adjustment of the timing of existing time points was made. The duration of sampling was not extended.

Following dosing on Day 1, blood samples were taken pre-dose (within one hour of dosing) and at 1, 2, 4, 6, 8 and 12 hours. Participants returned for a blood sample to be taken at 9 am in the morning on Days 2, 3, 4, 5, 8, 15, 22, 29, 43, 57, 71 and 85.

### 1.3 Results

Median Tₘₐₓ for plasma anastrozole was 2 days post-dose (range 1-3 days), with average Cₘₐₓ=5.30 ng/ml, and an average terminal half-life of 14 days. Concentrations were below the limit of quantitation (0.1 ng/mL) in most participants after 12 weeks. For serum testosterone, the median Tₘₐₓ was 8 hours post-dose (range 6-12 hours), with average Cₘₐₓ=4.1 ng/mL, compared with the average level of serum testosterone at baseline of 0.2 ng/mL. The observed increase in serum testosterone compared with baseline was sustained in all subjects at 12 weeks, with an average serum concentration of 0.8 ng/mL

Serum dihydrotestosterone (DHT) was not measurable in most samples, with Tₘₐₓ occurring at 2-3 days post-dose in participants with measurable maximal values. The measured serum levels of anastrozole, testosterone and DHT are set out in Tables 1-3 below.

TABLE 1
Measured serum anastrozole
Summary of Pharmacokinetic Concentration Over Time by Analyte
Analyte: Anastrozole

| | Concentration (ng/mL) | | | | | | | | | |
|---|---|---|---|---|---|---|---|---|---|---|
| Statistic/ | Pre- | Time post-dose (hours) | | | | | | | | |
| Subject | dose | 1 | 2 | 4 | 6 | 8 | 12 | 24 | 48 | 72 |
| S001 | BLQ | 0.215 | 0.404 | 1.03 | ND | 4.28 | 5.13 | 6.08 | 6.17 | 4.94 |
| S002 | BLQ | 0.525 | 0.615 | 1.00 | ND | 3.41 | 3.27 | 4.35 | 3.95 | 4.72 |
| S003 | BLQ | 0.218 | 1.30 | 2.97 | 3.11 | 3.48 | 4.42 | 4.29 | 4.94 | 5.16 |
| S004 | BLQ | 0.515 | 0.887 | 1.42 | 3.07 | 4.75 | 3.87 | 5.49 | 5.05 | 5.00 |
| S005 | BLQ | 0.394 | 0.831 | 2.23 | 3.42 | 4.16 | 4.32 | 5.20 | 5.85 | 6.29 |
| S009 | BLQ | 0.127 | 0.306 | 0.902 | 1.63 | 2.18 | 3.32 | 4.22 | 4.62 | 4.92 |
| S011 | BLQ | 0.855 | 1.04 | 1.50 | 3.08 | 3.50 | 4.32 | 4.96 | 5.58 | 4.60 |
| S012 | BLQ | 0.321 | 0.596 | 1.12 | 3.21 | 3.66 | 4.86 | 5.60 | 6.07 | 5.68 |
| S013 | BLQ | 0.243 | 0.509 | 0.582 | 1.62 | 3.33 | 4.18 | 4.66 | 4.85 | 4.76 |
| S014 | BLQ | BLQ | 0.180 | 0.260 | 0.651 | 1.06 | 2.25 | 3.08 | 3.31 | 3.66 |
| S015 | BLQ | 0.751 | 1.02 | 2.11 | 3.42 | 3.99 | 4.77 | 5.34 | 5.28 | 4.85 |
| N | 11 | 11 | 11 | 11 | 9 | 11 | 11 | 11 | 11 | 11 |
| Mean | 0.00 | 0.38 | 0.70 | 1.37 | 2.58 | 3.44 | 4.06 | 4.84 | 5.06 | 4.96 |
| Std Dev | 0.00 | 0.26 | 0.35 | 0.79 | 1.01 | 1.03 | 0.84 | 0.84 | 0.88 | 0.65 |
| CV (%) | 0% | 69% | 50% | 58% | 39% | 30% | 21% | 17% | 17% | 13% |
| Median | 0.00 | 0.32 | 0.62 | 1.12 | 3.08 | 3.50 | 4.32 | 4.96 | 5.05 | 4.92 |
| Minimum | 0.00 | 0.00 | 0.18 | 0.26 | 0.65 | 1.06 | 2.25 | 3.08 | 3.31 | 3.66 |
| Maximum | 0.00 | 0.86 | 1.30 | 2.97 | 3.42 | 4.75 | 5.13 | 6.08 | 6.17 | 6.29 |
| GeoMean | | 0.35 | 0.61 | 1.15 | 2.31 | 3.23 | 3.97 | 4.77 | 4.98 | 4.92 |

| | Concentration (ng/mL) | | | | | | | | |
|---|---|---|---|---|---|---|---|---|---|
| Statistic/ | Time post-dose (hours) | | | | | | | | |
| Subject | 96 | 168 | 336 | 504 | 672 | 1008 | 1344 | 1680 | 2016 |
| S001 | 4.36 | 2.84 | 1.93 | 1.70 | 0.809 | 0.332 | 0.144 | BLQ | BLQ |
| S002 | 4.12 | 3.23 | 1.92 | 1.42 | 0.922 | 0.665 | 0.279 | 0.143 | BLQ |
| S003 | 4.67 | 3.58 | 2.43 | 1.83 | 1.57 | 0.505 | BLQ | BLQ | BLQ |
| S004 | 5.17 | 4.35 | 2.64 | 1.59 | 1.27 | 0.624 | 0.358 | 0.195 | BLQ |
| S005 | 5.33 | 4.66 | 2.74 | 2.46 | 1.73 | 1.14 | 0.795 | 0.431 | 0.227 |
| S009 | 4.89 | 3.68 | 1.95 | 1.03 | 0.790 | 0.399 | 0.235 | BLQ | BLQ |
| S011 | 4.56 | 3.59 | 1.50 | 1.23 | 0.682 | 0.268 | BLQ | BLQ | BLQ |
| S012 | 5.70 | 4.89 | 2.40 | 1.25 | 0.712 | BLQ | BLQ | BLQ | BLQ |
| S013 | 4.19 | 2.93 | 1.70 | 1.19 | 0.767 | 0.311 | BLQ | BLQ | BLQ |
| S014 | 3.27 | 2.46 | 1.15 | 1.10 | 0.681 | 0.358 | 0.158 | BLQ | BLQ |
| S015 | 4.60 | 3.10 | 1.78 | 1.36 | 1.02 | 0.773 | 0.488 | 0.374 | 0.191 |
| N | 11 | 11 | 11 | 11 | 11 | 11 | 11 | 11 | 11 |
| Mean | 4.62 | 3.57 | 2.01 | 1.47 | 1.00 | 0.49 | 0.22 | 0.10 | 0.04 |
| Std Dev | 0.66 | 0.78 | 0.49 | 0.41 | 0.37 | 0.30 | 0.25 | 0.16 | 0.08 |
| CV (%) | 14% | 22% | 24% | 28% | 37% | 62% | 112% | 157% | 223% |
| Median | 4.60 | 3.58 | 1.93 | 1.36 | 0.81 | 0.40 | 0.16 | 0.00 | 0.00 |
| Minimum | 3.27 | 2.46 | 1.15 | 1.03 | 0.68 | 0.00 | 0.00 | 0.00 | 0.00 |
| Maximum | 5.70 | 4.89 | 2.74 | 2.46 | 1.73 | 1.14 | 0.80 | 0.43 | 0.23 |
| GeoMean | 4.58 | 3.50 | 1.95 | 1.42 | 0.94 | 0.49 | 0.30 | 0.26 | 0.21 |

Key:
BLQ = Below limit of Quantitation;
LLOQ = Limit of Quantification:
ND = Not Done
Note:
BLQ values set to zero for summary statistics, but excluded from calculation of geometric mean
LLOQ: Anastrozole = 0.1 ng/ml;
Testosterone = 0.02 ng/ml;
Dihydrotestosterone (DHT) = 0.04/0.1/0.2 ng/ml

TABLE 2
Measured serum testosterone
Summary of Pharmacokinetic Concentration Over Time by Analyte
Analyte: Testosterone

| | Concentration (ng/mL) | | | | | | | | | |
|---|---|---|---|---|---|---|---|---|---|---|
| Statistic/ | Pre- | Time post-dose (hours) | | | | | | | | |
| Subject | dose | 1 | 2 | 4 | 6 | 8 | 12 | 24 | 48 | 72 |
| S001 | 0.151 | 0.223 | 0.271 | 0.588 | ND | 3.18 | 3.05 | 2.27 | 1.61 | 1.29 |
| S002 | 0.219 | 0.665 | 0.500 | 0.960 | ND | 3.95 | 3.46 | 2.29 | 2.00 | 2.30 |
| S003 | 0.408 | 0.593 | 2.13 | 5.23 | 5.27 | 4.76 | 5.65 | 3.44 | 2.81 | 2.84 |
| S004 | 0.167 | 0.566 | 0.658 | 0.969 | 4.73 | 4.96 | 3.78 | 3.66 | 2.22 | 2.35 |
| S005 | 0.125 | 0.388 | 0.835 | 2.06 | 2.98 | 3.67 | 2.42 | 1.75 | 1.10 | 1.03 |
| S009 | 0.320 | 0.420 | 0.487 | 0.915 | 1.31 | 2.18 | 3.32 | 2.12 | 1.72 | 1.32 |
| S011 | 0.336 | 1.01 | 0.736 | 0.709 | 4.27 | 3.05 | 2.95 | 2.43 | 1.81 | 1.60 |
| S012 | 0.187 | 0.467 | 0.514 | 0.887 | 2.66 | 3.08 | 3.40 | 2.21 | 1.61 | 1.32 |
| S013 | 0.147 | 0.377 | 0.299 | 0.237 | 1.54 | 4.15 | 3.22 | 1.64 | 1.28 | 0.929 |
| S014 | 0.204 | 0.237 | 0.312 | 0.212 | 0.526 | 1.04 | 3.23 | 2.50 | 1.63 | 2.24 |
| S015 | 0.088 | 1.08 | 1.28 | 2.93 | 4.78 | 5.08 | 3.29 | 2.67 | 2.35 | 1.82 |
| N | 11 | 11 | 11 | 11 | 9 | 11 | 11 | 11 | 11 | 11 |
| Mean | 0.21 | 0.55 | 0.73 | 1.43 | 3.12 | 3.55 | 3.43 | 2.45 | 1.83 | 1.73 |
| Std Dev | 0.10 | 0.28 | 0.55 | 1.49 | 1.73 | 1.23 | 0.81 | 0.62 | 0.49 | 0.62 |
| CV (%) | 47% | 51% | 75% | 104% | 56% | 35% | 24% | 25% | 27% | 36% |
| Median | 0.19 | 0.47 | 0.51 | 0.92 | 2.98 | 3.67 | 3.29 | 2.29 | 1.72 | 1.60 |
| Minimum | 0.09 | 0.22 | 0.27 | 0.21 | 0.53 | 1.04 | 2.42 | 1.64 | 1.10 | 0.93 |
| Maximum | 0.41 | 1.08 | 2.13 | 5.23 | 5.27 | 5.08 | 5.65 | 3.66 | 2.81 | 2.84 |
| GeoMean | 0.19 | 0.49 | 0.60 | 0.94 | 2.53 | 3.29 | 3.36 | 2.39 | 1.77 | 1.63 |

| | Concentration (ng/mL) | | | | | | | | |
|---|---|---|---|---|---|---|---|---|---|
| Statistic/ | Time post-dose (hours) | | | | | | | | |
| Subject | 96 | 168 | 336 | 504 | 672 | 1008 | 1344 | 1680 | 2016 |
| S001 | 1.05 | 0.810 | 1.55 | 1.51 | 1.06 | 0.672 | 1.09 | 0.791 | 0.592 |
| S002 | 1.89 | 1.63 | 1.77 | 1.84 | 1.66 | 1.17 | 1.07 | 1.11 | 0.911 |
| S003 | 2.01 | 2.47 | 2.49 | 3.30 | 2.99 | 1.75 | 1.55 | 0.995 | 0.908 |
| S004 | 2.17 | 2.33 | 2.18 | 2.27 | 1.48 | 1.23 | 0.962 | 1.07 | 0.804 |
| S005 | 1.01 | 1.06 | 2.46 | 2.17 | 1.65 | 1.09 | 1.10 | 0.927 | 0.686 |
| S009 | 1.35 | 0.839 | 1.40 | 2.24 | 2.00 | 1.79 | 1.76 | 1.19 | 0.925 |
| S011 | 1.88 | 1.87 | 1.70 | 2.09 | 1.92 | 1.40 | 1.42 | 1.03 | 0.849 |
| S012 | 1.34 | 1.39 | 2.76 | 1.91 | 1.49 | 1.48 | 1.02 | 0.975 | 0.970 |
| S013 | 0.874 | 1.10 | 1.90 | 1.48 | 1.44 | 1.29 | 1.14 | 0.795 | 0.659 |
| S014 | 1.63 | 1.46 | 2.41 | 2.57 | 1.78 | 1.61 | 1.19 | 0.897 | 0.595 |
| S015 | 1.87 | 1.89 | 4.17 | 3.50 | 2.94 | 2.81 | 1.46 | 1.17 | 1.19 |
| N | 11 | 11 | 11 | 11 | 11 | 11 | 11 | 11 | 11 |
| Mean | 1.55 | 1.53 | 2.25 | 2.26 | 1.86 | 1.48 | 1.25 | 1.00 | 0.83 |
| Std Dev | 0.45 | 0.57 | 0.77 | 0.65 | 0.60 | 0.54 | 0.26 | 0.14 | 0.18 |
| CV (%) | 29% | 37% | 34% | 29% | 33% | 37% | 20% | 14% | 22% |
| Median | 1.63 | 1.46 | 2.18 | 2.17 | 1.66 | 1.40 | 1.14 | 1.00 | 0.85 |
| Minimum | 0.87 | 0.81 | 1.40 | 1.48 | 1.06 | 0.67 | 0.96 | 0.79 | 0.59 |
| Maximum | 2.17 | 2.47 | 4.17 | 3.50 | 2.99 | 2.81 | 1.76 | 1.19 | 1.19 |
| GeoMean | 1.49 | 1.44 | 2.15 | 2.18 | 1.78 | 1.40 | 1.23 | 0.99 | 0.81 |

Key:
BLQ = Below Limit of Quantitation;
LLOQ = Lower Limit of Quantification:
ND = Not Done
Note:
BLQ values set to zero for summary statistics, but excluded from calculation of geometric mean
LLOQ: Anastrozole = 0.1 ng/ml;
Testosterone = 0.02 ng/ml;
Dihydrotestosterone (DHT) = 0.04/0.1/0.2 ng/ml

TABLE 3
Measured serum dihydrotestosterone
Summary of Pharmacokinetic Concentration Over Time by Analyte
Analyte: Dihydrotestosterone

| | Concentration (ng/ml) | | | | | | | | | |
|---|---|---|---|---|---|---|---|---|---|---|
| Statistic/ | Pre- | Time post-dose (hours) | | | | | | | | |
| Subject | dose | 1 | 2 | 4 | 6 | 8 | 12 | 24 | 48 | 72 |
| S001 | 0.088 | 0.084 | 0.076 | 0.100 | ND | 0.123 | 0.126 | 0.201 | 0.227 | 0.142 |
| S002 | 0.172 | 0.126 | 0.088 | 0.187 | ND | 0.193 | 0.197 | 0.251 | 0.242 | 0.285 |
| S003 | BLQ | BLQ | BLQ | BLQ | BLQ | BLQ | BLQ | BLQ | 0.222 | BLQ |
| S004 | BLQ | BLQ | BLQ | BLQ | BLQ | BLQ | BLQ | BLQ | BLQ | BLQ |
| S005 | BLQ | BLQ | BLQ | BLQ | BLQ | BLQ | BLQ | BLQ | BLQ | BLQ |
| S009 | BLQ | BLQ | BLQ | BLQ | BLQ | BLQ | BLQ | BLQ | BLQ | BLQ |
| S011 | BLQ | BLQ | BLQ | BLQ | BLQ | BLQ | BLQ | BLQ | BLQ | BLQ |
| S012 | BLQ | BLQ | BLQ | BLQ | BLQ | BLQ | BLQ | BLQ | BLQ | BLQ |
| S013 | BLQ | BLQ | BLQ | BLQ | BLQ | BLQ | BLQ | BLQ | BLQ | BLQ |
| S014 | BLQ | BLQ | BLQ | 0.987 | BLQ | BLQ | BLQ | BLQ | BLQ | BLQ |
| S015 | BLQ | BLQ | BLQ | BLQ | BLQ | BLQ | BLQ | BLQ | BLQ | BLQ |
| N | 11 | 11 | 11 | 11 | 9 | 11 | 11 | 11 | 11 | 11 |
| Mean | 0.02 | 0.02 | 0.01 | 0.12 | 0.00 | 0.03 | 0.03 | 0.04 | 0.06 | 0.04 |
| Std Dev | 0.06 | 0.04 | 0.03 | 0.30 | 0.00 | 0.07 | 0.07 | 0.09 | 0.11 | 0.09 |
| CV (%) | 236% | 228% | 223% | 255% | 0% | 229% | 229% | 224% | 171% | 237% |
| Median | 0.00 | 0.00 | 0.00 | 0.00 | 0.00 | 0.00 | 0.00 | 0.00 | 0.00 | 0.00 |
| Minimum | 0.00 | 0.00 | 0.00 | 0.00 | 0.00 | 0.00 | 0.00 | 0.00 | 0.00 | 0.00 |
| Maximum | 0.17 | 0.13 | 0.09 | 0.99 | 0.00 | 0.19 | 0.20 | 0.25 | 0.24 | 0.29 |
| GeoMean | 0.12 | 0.10 | 0.08 | 0.26 | ND | 0.15 | 0.16 | 0.22 | 0.23 | 0.20 |

| | Concentration (ng/ml) | | | | | | | | |
|---|---|---|---|---|---|---|---|---|---|
| Statistic/ | Time post-dose (hours) | | | | | | | | |
| Subject | 96 | 168 | 336 | 504 | 672 | 1008 | 1344 | 1680 | 2016 |
| S001 | 0.129 | 0.137 | 0.066 | 0.118 | 0.085 | BLQ | BLQ | BLQ | BLQ |
| S002 | 0.231 | 0.254 | 0.229 | 0.245 | 0.190 | BLQ | BLQ | BLQ | BLQ |
| S003 | BLQ | BLQ | BLQ | BLQ | BLQ | BLQ | BLQ | BLQ | BLQ |
| S004 | BLQ | BLQ | BLQ | BLQ | BLQ | BLQ | BLQ | BLQ | BLQ |
| S005 | BLQ | BLQ | BLQ | BLQ | BLQ | BLQ | BLQ | BLQ | BLQ |
| S009 | BLQ | BLQ | BLQ | BLQ | BLQ | BLQ | 0.136 | 0.119 | 0.108 |
| S011 | 0.254 | BLQ | BLQ | BLQ | BLQ | BLQ | 0.172 | 0.107 | 0.108 |
| S012 | BLQ | BLQ | BLQ | BLQ | BLQ | BLQ | 0.182 | 0.152 | 0.212 |
| S013 | BLQ | BLQ | BLQ | BLQ | BLQ | BLQ | BLQ | BLQ | BLQ |
| S014 | BLQ | BLQ | BLQ | BLQ | BLQ | BLQ | BLQ | BLQ | BLQ |
| S015 | BLQ | BLQ | BLQ | BLQ | BLQ | BLQ | BLQ | BLQ | BLQ |
| N | 11 | 11 | 11 | 11 | 11 | 11 | 11 | 11 | 11 |
| Mean | 0.06 | 0.04 | 0.03 | 0.03 | 0.03 | 0.00 | 0.04 | 0.03 | 0.04 |
| Std Dev | 0.10 | 0.08 | 0.07 | 0.08 | 0.06 | 0.00 | 0.08 | 0.06 | 0.07 |
| CV (%) | 179% | 234% | 261% | 239% | 241% | 0% | 173% | 174% | 185% |
| Median | 0.00 | 0.00 | 0.00 | 0.00 | 0.00 | 0.00 | 0.00 | 0.00 | 0.00 |
| Minimum | 0.00 | 0.00 | 0.00 | 0.00 | 0.00 | 0.00 | 0.00 | 0.00 | 0.00 |
| Maximum | 0.25 | 0.25 | 0.23 | 0.25 | 0.19 | 0.00 | 0.18 | 0.15 | 0.21 |
| GeoMean | 0.20 | 0.19 | 0.12 | 0.17 | 0.13 | | 0.16 | 0.12 | 0.14 |

Key:
BLQ = Below Limit of Quantitation;
LLOQ = Lower Limit of Quantification:
ND = Not Done
Note:
BLQ values set to zero for summary statistics, but excluded from calculation of geometric mean
LLOQ: Anastrozole = 0.1 ng/ml;
Testosterone = 0.02 ng/ml;
Dihydrotestosterone (DHT) = 0.04/0.1/0.2 ng/ml

Measured serum estradiol did not fall in the testosterone and anastrozole treatment group. This is consistent with the low level of anastrozole utilized in the study, which is 5 times lower than that achieved with conventional full oral dosing.

### 2. Effect of DHT on Breast Cancer Tissue

To evaluate the effect of DHT on breast cancer tissue, 3 primary tumours were examined in the presence of a low anastrozole level of 5 nM employing a modification of the method described by Jankovic-Karasoulos T. et al., 2008.

Briefly, the tumour tissue was cut into 3 mm² pieces and placed in triplicate on gelatin sponges submersed in media supplemented with 10% dextran-coated charcoal stripped foetal calf serum (DCC-FCS) containing vehicle (control) or either 0.1, 1 or 10 nM (nmol/l) DHT to replicate DHT level in breast tissue correlating to increasing serum testosterone level.

The 3 explant invasive breast carcinomas were examined for expression of mTOR and c-Myc and response to exposure to DHT by Western blotting. Whole cell lysates of tumour and control adipose tissue were prepared by sonication at 48° C. in lysis buffer (1% Triton X-100, 50 mM KCl, 25 mM HEPES, pH 7.8, 10 mg/ml of leupeptin, 20 mg/ml of aprotinin, 125 mM dithiothreitol and 1 mM phenylmethylsulfonyl fluoride) and analyzed on the same Western blot. 50 mg samples of total protein were mixed with 50 ml of sodium dodecyl sulfate (SDS)-mercaptoethanol sample buffer and boiled for 10 min, then the proteins were separated on 7.5% SDS gels and transferred to a polyvinylidene fluoride membrane. The membrane was then blocked for 1 h at room temperature using 5% skimmed milk in phosphate-buffered saline (PBS) containing 0.5% Tween-20, immunoblotted with antibodies against human mTOR or MYC protein diluted in PBS and horseradish peroxidase-conjugated secondary antibodies (Novus Biologicals LLC CO USA) diluted in PBS, followed by detection with Chemiluminescence Reagent (Amersham Bioscience, Buckinghamshire, England). The band density was measured by densitometry, using Image Master VDS and Image Quant Analysis Software (Amersham Pharmacia Biotech, Hong Kong). The relative protein levels of mTOR or MYC and b-actin in the original total protein lysate from the breast preparations were obtained. mTOR or MYC protein expression was normalized to beta-actin protein expression. Immunizing host antibodies were produced with a synthetic peptide derived from the sequence of human mTOR or MYC, purified by peptide affinity chromatography and confirmed using control peptides. The results of the Western blot analysis for mTOR or MYC proteins in the 3 explant samples at baseline and after 24 hours of culture are shown in Table 3 and Table 4 for mTOR or MYC respectively. Cell lysates were immunoblotted with antibodies to mTOR or MYC. The experiment was performed twice with similar results. Data are expressed relative to beta-actin.

TABLE 3
mTOR Western blot ratio to beta-Actin

| Patient | Pre- | 0.1 nM | 1 nM | 10 nM |
|---|---|---|---|---|
| number | treatment | DHT | DHT | DHT |
| 1 | 0.35 | 0.81 | 0.04 | 0.03 |
| 2 | 0.38 | 1.01 | 0.02 | 0.05 |
| 3 | 0.42 | 1.23 | 0.06 | 0.04 |
| Average | 0.38 | 1.01 | 0.04 | 0.04 |

TABLE 4
MYC Western blot ratio to beta-Actin

| Patient | Pre- | 0.1 nM | 1 nM | 10 nM |
|---|---|---|---|---|
| number | treatment | DHT | DHT | DHT |
| 1 | 0.40 | 0.39 | 0.04 | 0.03 |
| 2 | 0.50 | 1.01 | 0.02 | 0.05 |
| 3 | 0.50 | 1.23 | 0.06 | 0.04 |
| Average | 0.45 | 1.01 | 0.04 | 0.04 |

It has recently been demonstrated that a tumoural DHT level of from 1 nM up to an evaluated level of 10 nM can induce androgen receptor (AR) to become a tumour suppressor of estrogen receptor (ER) positive breast cancer (Hickey T E. et al., 2021). In that study, a diverse, clinically relevant panel of cell-line and patient-derived model was used to demonstrate that AR activation, not suppression, exerts potent antitumor activity in multiple disease contexts, including resistance to standard-of-care ER and CDK4/6 inhibitors. Notably, AR agonists combined with standard-of-care agents were found to enhance therapeutic responses. Mechanistically, agonist activation of AR altered the genomic distribution of estrogen receptor (ER) and essential co-activators (p300, SRC-3), resulting in repression of ER-regulated cell cycle genes and upregulation of AR target genes, including known tumor suppressors.

In the present study, mTOR and Myc were found to be markedly elevated in the presence of 0.1 nM DHT, barely discernable in breast cancer explant tissue treated with 1 nM DHT and essentially not detected in tissue treated with 10 nM DHT.

### 3. Discussion

The present results show a unique combination of medium dose testosterone with low dose aromatase inhibitor can induce an intramammary and intratumoural level of dihydrotestosterone that avoids or minimizes induction of the mTOR pathway associated with treatment with 0.1 nM DHT and which is known to be associated with increased proliferation and dedifferentiation of malignancy. Relevantly, the results further show an intra-tumoural concentration of dihydrotestosterone of from 1 to 10 nM results in suppression of c-Myc, the well-established generator of cancer progression and insensitivity to both classical hormonal therapies as well as other therapies not considered to be hormonal breast cancer therapies, including those that act on the HER2 and nitric oxide pathways. In contrast, 0.1 nM DHT was unable to suppress c-Myc.

Increasing DHT in the breast can result in down regulation of aromatase in breast tissue (Perel E. et al.,) and up regulation of 5α-reductase (McNamara K M. et al., 2014) to provide an antiestrogenic affect. However, the present results show that this can only occur when the intramammary concentration of DHT is at a sufficient level for inducing suppression of c-Myc without induction of mTOR. Without being limited by theory, this is believed by the inventor to be the result of AR dimer formation and transcriptional activation of AR at the higher levels of DHT evaluated (i.e., from 1-10 nM), whereas activation of c-Myc and mTOR activity is associated with expression of AR in monomer form at the lower concentration of 0.1 nM DHT.

The consequence of a combination treatment embodied by the present disclosure alone or in combination with other breast cancer therapeutics is the perpetuation of a differentiated therapeutically responsive phenotype of malignancy or potentially malignant phenotype on the spectrum from initiation, promotion and advanced breast cancer, reducing the risk of progression to hormonal resistance cancer or cancer insensitive to targeted drug therapy as herein described.

It has previously been demonstrated that intramammary and intratumoural testosterone levels are in equilibrium with serum levels whereas due to induction of 5α-reductase, DHT levels in tumour tissue is at least 3 time higher than that observed in the serum (Recchione C. et al, 1995). On the basis of the present results, at least 1 nM DHT is required for suppression of cMyc and to inhibit induction of mTOR activity via non-genomic interaction. Further, intratumoural levels of DHT in the women in the present pharmacokinetic study would be expected to achieve a peak of 14 nM for a short duration essentially without elevating serum levels of DHT, thus avoiding virilization yet achieving intramammary androgenization as per Raths F. et al., 2023).

Methods of the present disclosure allow for ongoing treatment of a subject by periodic repeat administration of androgenic agent and aromatase inhibitor combination therapy as described herein, typically by subcutaneous pellet at 3-month intervals. After 3 months post administration when the subject's testosterone level troughs the intramammary concentration of DHT would nevertheless be above approx. 2 nM with continued suppression of c-Myc expression and mTOR activity associated with lower DHT concentration as described above. Repeat administration of the combination subcutaneous implant therefore maintains the subject's intramammary and intratumoural testosterone and DHT levels within the desired therapeutic range for prophylaxis or treatment of the premalignant or malignant breast tissue pathology and inhibition of progression to hormone resistant or targeted drug insensitive breast cancer, substantially without increasing serum DHT levels in the systemic circulation as described herein.

Various modifications may be made to embodiments as described herein without departing from the scope of the present disclosure and the above examples are to be taken as illustrative only and not restrictive.

### REFERENCES CITED

Deshpander, N., P. Carson, I. Mitchell and B. Berry, (1977). The accumulation of 5alpha-dihydrotesterone (5alpha-DHT) by human breast tissue. J Steroid Biochem, 8 (1): 105-106.
Elshazly, A. M. and D. A. Gewirtz, (2022). An overview of resistance to Human epidermal growth factor receptor 2 (Her2) targeted therapies in breast cancer. Cancer Drug Resist 5 (2): 472-486.
Glaser R and Dimitrakakis C, (2021). Testosterone Implant Therapy in Women With and Without Breast Cancer: Rationale, Experience, Evidence. Androgens: Clinical Research and Therapeutics, Vol. 2.1, pp. 94-110.
Han W et al., (2022). Exploiting the tumor-suppresive activity of the androgen receptor by CDK4/6 inhibition in castration-resistant prostate cancer. Molecular Therapy Vol. 30, No 4, April, pp. 1628-1644.
Hickey, T. E., L. A. Selth, K. M. Chia, G. Laven-Law, H. H. Milioli, D. Roden, S. Jindal, M. Hui, J. Finlay-Schultz, E. Ebrahimie, S. N. Birrell, S. Stelloo, R. Iggo, S. Alexandrou, C. E. Caldon, T. M. Abdel-Fatah, I. O. Ellis, W. Zwart, C. Palmieri, C. A. Sartorius, A. Swarbrick, E. Lim, J. S. Carroll and W. D. Tilley, (2021). The androgen receptor is a tumor suppressor in estrogen receptor-positive breast cancer. Nat Med 27 (2): 310-320.
Hong, R., Cao, B., Chen, D., Wu, W., Luo, T., Lv, D., Zhang, W., Wang, S. and Shao, K (2024). Multi-omics portrait of ductal cell carcinoma in situ in young women. Breast Cancer Res and Treatment. Springer. Published on-line 5 May 2024. doi.org/10.1007/s10549-024-07254-5.
Hosseinzadeh, L., Z. Kikhtyak, G. Laven-Law, S. M. Pederson, C. G. Puiu, C. S. D'Santos, E. Lim, J. S. Carroll, W. D. Tilley, A. R. Dwyer and T. E. Hickey (2024). The androgen receptor interacts with GATA3 to transcriptionalluy regulate a luminal epithelial cell phenotype in breast cancer. Genome Biol 25 (1): 44, pp 1-28.
Jankovic-Karasoulos, T., S. Birrell, E. Cops, S. Jindal, A. Ochnik, M. Thomas, W. Tilley and T. Hickey, (2009). Evaluation of Testosterone Supplementation during Anastrozole Therapy in a Breast Explant Model. Cancer Research 69 (24_Supplement): 4089-4089.
Ji W et al., (2020). c-myc regulates the sensitivity of breast cancer cells to palbociclib via c-myc/miR-29b-3p/CDK6 axis. Cell Death and Disease., 11:760, pp 1-13.
Li, Y., Y. Guo, F. Chen, Y. Cui, X. Chen and G. Shi, (2024). Male breast cancer differs from female breast cancer in molecular features that affect prognoses and drug responses. Transl_Oncol 45:101980, pp. 1-18.
McNamara, K. M., T. Yoda, A. M. Nurani, Y. Shibahara, Y. Miki, L. Wang, Y. Nakamura, K. Suzuki, Y. Yang, E. Abe, H. Hirakawa, T. Suzuki, N. Nemoto, M. Miyashita, K. Tamaki, T. Ishida, K. A. Brown, N. Ohuchi and H. Sasano, (2014). Androgenic pathways in the progression of triple-negative breast carcinoma: a comparison between aggressive and non-aggressive subtypes. Breast Cancer Res Treat 145 (2): 281-293.
Perel, E., K. H. Stolee, L. Kharlip, M. E. Blackstein and D. W. Killinger, (1984). The intracellular control of aromatase activity by 5 alpha-reduced androgens in human breast carcinoma cells in culture. J Clin Endocrinol Metab 58 (3): 467-472.
Raths, F., M. Karimzadeh, N. Ing, A. Martinez, Y. Yang, Y. Qu, T. Y. Lee, B. Mulligan, S. Devkota, W. T. Tilley, T. E. Hickey, B. Wang, A. E. Giuliano, S. Bose, H. Goodarzi, E. C. Ray, X. Cui and S. R. V. Knott, (2023). The molecular consequences of androgen activity in the human breast. Cell Genom 3 (3): 100272.
Recchione, C., E. Venturelli, A. Manzari, A. Cavalleri, A. Martinetti and G. Secreto, (1995). Testosterone, dihydrotestosterone and oestradiol levels in postmenopausal breast cancer tissues. J Steroid Biochem Mol Biol 52 (6): 541-546.
Qiu, X et al., (2022). MYC drives aggressive prostate cancer by disrupting transcriptional pause release at androgen receptor targets. Nature Communications, 13:2559, pp. 1-17.

1. A method for prophylaxis or treatment of an aberrant breast cell pathology characterized by cell proliferation in a subject, the method comprising administering an effective amount of an androgenic agent in combination with an effective amount of an aromatase inhibitor to the subject providing an increase in concentration of the androgenic agent in blood serum to a level for increasing intramammary concentration of an androgen in the breast arising from action of 5α-reductase enzyme on the androgenic agent, the increased intramammary concentration of the androgen inducing suppression of c-Myc and/or mTOR activity in the breast associated with a lower intramammary concentration of the androgen, and wherein the increase in the serum concentration of the androgenic agent and increase in intramammary concentration of the androgen is maintained for a predetermined period substantially without increasing concentration of the androgen in the serum of the subject.

2. The method of claim 1, wherein the aromatase inhibitor blocks aromatization of the androgenic agent to an oestrogen.

3. The method according to claim 1, wherein there is substantially no increase in oestrogen concentration in the breast during the predetermined period.

4. (canceled)

5. The method of claim 1, wherein the androgen is 5α-dihydrotestosterone (DHT) or a physiologically active form of DHT.

6. The method of claim 1, wherein the lower intramammary concentration of the androgen is 1 nM.

7-8. (canceled)

9. The method of claim 1, wherein the androgenic agent is selected from testosterone and physiologically active forms of testosterone.

10. The method of claim 1, wherein the androgenic agent is testosterone.

11. The method of claim 1, wherein the aromatase inhibitor is selected from the group consisting of anastrozole, letrozole, fadrozol, vorozole, exemestane and formestane.

12. The method of claim 1, wherein the androgen is DHT.

13. (canceled)

14. The method of claim 1, wherein the androgenic agent is testosterone and the increased concentration of testosterone in the blood serum of the subject is at least 1 ng/ml.

15. (canceled)

16. The method of claim 1, wherein the androgenic agent and the aromatase inhibitor are administered to the subject by subcutaneous, intramuscular and/or transdermal application for sustained delivery of the androgenic agent and the aromatase inhibitor to the subject.

17. The method of claim 1, wherein the androgenic agent and the aromatase inhibitor are administered to the subject by subcutaneous implant.

18. The method of claim 1, wherein the increase in serum concentration of the androgenic agent is maintained for the predetermined period of at least 1 week.

19-20. (canceled)

21. The method of claim 1, wherein the suppression of c-Myc and/or mTOR activity comprises substantially complete suppression of c-Myc activity and/or mTOR activity.

22. The method of claim 1, wherein the androgen receptor (AR) is expressed at least primarily as an AR monomer in the breast below the lower intramammary concentration of the androgen and at least primarily in AR dimer form above the lower intramammary concentration of the androgen.

23. The method of claim 1, wherein the aberrant breast cell pathology is breast cancer.

24. The method of claim 23, wherein the breast cancer is a pre-invasive breast cancer.

25. The method of claim 24, wherein the breast cancer is malignant breast cancer.

26. The method of claim 24 wherein the breast cancer is ductal or lobular cell carcinoma in situ.

27. The method of claim 1, wherein the aberrant breast cell pathology is breast cell hyperplasia.

28. The method of claim 1, wherein the breast cell hyperplasia is lobular cell hyperplasia.

29. The method of claim 1, wherein the risk of the aberrant breast cell pathology progressing to hormonal resistant breast cancer or breast cancer insensitive to targeted HER2 anti-cancer drug therapy is reduced.

30. A method for reducing risk of breast cancer in a subject progressing to hormone resistant breast cancer, the method comprising administering an effective amount of an androgenic agent in combination with an effective amount of an aromatase inhibitor to the subject providing an increase in concentration of the androgenic agent in blood serum to a level for increasing intramammary concentration of the androgen in the breast arising from action of 5α-reductase enzyme on the androgenic agent, the increased intramammary concentration of the androgen inducing suppression of c-Myc and/or mTOR activity in the breast associated with a lower intramammary concentration of the androgen, and wherein the increase in the serum concentration of the androgenic agent and increase in intramammary concentration of the androgen in the breast is maintained for a predetermined period substantially without increasing concentration of the androgen in the serum of the subject.

31. A method for reducing risk of HER2-positive breast cancer in a subject becoming insensitive to targeted HER2 anti-cancer drug therapy, the method comprising administering an effective amount of an androgenic agent in combination with an effective amount of an aromatase inhibitor to the subject providing an increase in concentration of the androgenic agent in blood serum to a level for increasing intramammary concentration of the androgen in the breast arising from action of 5α-reductase enzyme on the androgenic agent, the increased intramammary concentration of the androgen inducing suppression of c-Myc and/or mTOR activity in the breast associated with a lower intramammary concentration of the androgen, and wherein the increase in the serum concentration of the androgenic agent and increase in intramammary concentration of the androgen in the breast is maintained for a predetermined period substantially without increasing concentration of the androgen in the serum of the subject.

32-33. (canceled)

34. A method for inducing suppression of c-Myc and/or mTOR activity in breast of a subject, the method comprising administering an effective amount of an androgenic agent in combination with an effective amount of an aromatase inhibitor to the subject providing an increase in concentration of the androgenic agent in blood serum and an increase in intramammary concentration of an androgen to induce suppression of c-Myc and/or mTOR activity in the breast associated with a lower intramammary concentration of the androgen, the increase in concentration of the androgen arising from the action of 5α-reductase enzyme on the androgenic agent.

35. The method of claim 1, wherein the androgen is 5-dihydrotestosterone (DHT) or a physiologically active form of DHT, and wherein the DHT or physiologically active form of DHT is increased to above 1 nM for the predetermined period.
